(19) United States
(12) Patent Application Publication
(10) Pub. No.: US 2025/0101233 A1
(43) Pub. Date: Mar. 27, 2025

(54) STABLE NATURAL COLOURANT

(71) Applicant: Givaudan SA, VERNIER (CH)

(72) Inventors: Luciana Andreia DE SOUZA CUNHA, Botucatu (BR); Fabio MAGALHAES DE MELLO, Sao Paolo (BR); Fabienne BOUKOBZA-RAVEY, Bedford Park, IL (US); Altieres ALMEIDA, Botucatu (BR); Reginaldo GUILHERME, Botucatu (BR)

(21) Appl. No.: 18/730,783

(22) PCT Filed: Jan. 27, 2023
(86) PCT No.: PCT/EP2023/051980

§ 371 Date: Jul. 21, 2024

Related U.S. Application Data
(60) Provisional application No. 63/305,904, filed on Feb. 2, 2022.

(51) Int. Cl. C09B 61/00

(52) U.S. Cl. CPC C09B 61/00

(57) ABSTRACT

The present invention relates to stable black and brown compositions and colourants comprising an extract obtained or obtainable from genipap juice. The present invention further relates to the uses of these compositions and colourants, methods of making the compositions and colourants and food products comprising the compositions and colourants.

### FIELD OF THE INVENTION

The present invention relates to compositions and colourants comprising an extract obtained or obtainable from genipap juice rich in genipin and an extract comprising proteins, polypeptides and/or amino acids obtained or obtainable from white beans, soy, pea, chickpea, lentil, milk or mixtures thereof. The present invention further relates to the uses of these compositions and colourants, methods of making the compositions and colourants and food products comprising the compositions and colourants.

U.S. 63/305,904 is incorporated by reference for all that it contains.

### BACKGROUND OF THE INVENTION

It is becoming increasingly more desirable to colour food and cosmetic products using natural or organic based food colourants in place of synthetic colourants.

The fruit genipa (Genipa americana L) is a fruit native to Brazil and has been used as a source for obtaining a natural blue colourant.

Genipa americana L contains genipin and other irioid compounds, such as genipic acid, genipin gentioside, geniposide and geniposidic acid.

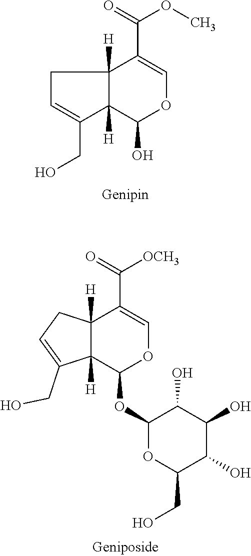 [SMILES: COC(=O)C1=CO[C@@H](O)[C@@H]2C(CO)=CC[C@H]12 ; COC(=O)C1=CO[C@@H](O[C@@H]2O[C@H](CO)[C@@H](O)[C@H](O)[C@H]2O)[C@@H]2C(CO)=CC[C@H]12]

Genipa americana is also a natural source of iron, riboflavin, and anti-bacterial substances, apart from the carbohydrates, sugar, proteins, ash, and malic acid found in its fruits. The principal biochemical compounds of Genipa americana include: calcium, phosphorous, vitamin C, caffeine, caterine, genipic acid, genipin, gentiobioside, genipinic acid, geniposide, geniposidic acid, gardenoside, genamesides A-D, gardendiol, deacetyl asperulosidic acid methyl ester, shanzhiside, glycerides, hydantoin, mannitol, methyl esters, tannic and tartaric acid, and tannins.

However, the blue colour provided by Genipa americana L has been found to be unstable, in particular, to heat. Thus, the use of Genipa americana L as a natural blue colourant has limitations in products that require heating, such as some food products.

The present invention seeks to address the abovementioned problems associated with instability of the blue colour provided by Genipa americana L and provide a composition and/or colourant that is stable, in particular, heat stable.

The present invention also provides novel brown and black colours obtained from genipa juice, and a process for preparing these colours.

### DISCLOSURE OF THE INVENTION

Methods for the Preparation of Compositions and/or Colourants of the Invention

As noted above, an advantageous aspect of the present invention is that the composition and/or colourant may be prepared using a very simple process with minimal steps. As will be appreciated by the person skilled in the art, simplification and/or reduction of process steps is advantageous as it reduces process time, costs and/or the need to specific equipment to perform the process.

Thus, the present invention provides a method of providing a composition and/or colourant (in particular, a composition that provides a blue or black colour or a blue or black colourant), wherein the method comprises:
(i) contacting genipin with oxygen;
(ii) combining the product from step (i) with a composition comprising amino acids, polypeptides and/or proteins (or CCP), and optionally
(iii) filtering the mixture obtained in step (ii) to remove insoluble solids.

The present invention provides a method for providing a black composition/colourant, wherein the method comprises:
(i) contacting genipin with oxygen;
(ii) combining the product from step (i) with a composition comprising amino acids, polypeptides, and/or proteins;
and optionally
(iii) filtering the mixture obtained in step (ii) to remove insoluble solids, wherein the product from step (i) and the protein, peptide and/or polypeptide of the composition comprising amino acids, polypeptides, and/or proteins is combined in a ratio of from about 0.5:5 to about 1:10, such as from about 0.5:4 to about 0.5:8 and the colour provided by the composition and/or colourant is black.

The composition comprising amino acids, polypeptides and/or proteins (or CCP) includes but is not limited to an extract obtained or obtainable from fruits, vegetables, grains, legumes, nuts, seeds, plant materials, animal materials including milk and eggs, microbial and algal materials, by-products from such sources and mixtures thereof.

Such method is hereinafter referred to as the “method of the invention”.

In the method of the invention, the genipin may be obtained from a natural source or may be synthetic.

Any genipin containing natural source may be used in the present invention.

In certain embodiments, the genipin may be from an extract obtained or obtainable from genipa (Genipa americana L). In the present disclosure, “genipa” and “Genipa americana L” are refereeing to the same fruit and are interchangeable.

Thus, the present invention provides a method of providing a composition and/or colourant (in particular, a composition that provides a blue or black colour or a blue or black colourant), wherein the method comprises:
(i) contacting a Genipa americana L extract comprising genipin with oxygen;
(ii) combining the product from step (i) with a composition comprising amino acids, polypeptides and/or proteins (or CCP) and optionally
(iii) filtering the mixture obtained in step (ii) to remove insoluble solids.

The genipin obtained or obtainable from the Genipa americana L may be extracted by any method know in the art. In certain embodiments, the genipin obtained or obtainable from the Genipa americana L may be purified. In certain embodiments the Genipa americana L extract comprising genipin used in the present invention has a concentration of genipin of at least 0.5% w/w, such as at least 0.67% w/w genipin, such as at least 1% w/w, at least 5% w/w, at least 10% w/w, at least 15% w/w, at least 20% w/w, at least 50% w/w genipin.

In certain embodiments, the Genipa americana L extract comprising genipin used in the present invention is highly purified. By highly purified in the present invention is mean an extract comprising at least 90% w/w of genipin, such as at least 99% of genipin.

In certain embodiments, the Genipa americana L extract comprising genipin used in the present invention comprises at least 0.5% w/w, such as at least 0.67% w/w genipin, such as at least 1% w/w, at least 5% w/w, at least 10% w/w, at least 15% w/w, at least 20% w/w, at least 50% w/w genipin.

In certain embodiments, the Genipa americana L extract comprising genipin may be obtained by pressing the fruit, i.e. the extract obtained or obtainable from Genipa americana L may be Genipa americana L fruit juice.

The fruit may be pressed by any suitable means. For example, the fruit may be pressed in a continuous press.

Typically, the whole fruit may be pressed. That is, the fruit including the skin and seeds.

After pressing, any insoluble solid may be discarded from the juice. For example, the skin and/or fruit pomace and/or seeds may be discarded.

After pressing the juice may be sieved. For example, the juice may be sieved through a 30 to 60 mesh sieve, i.e., a stainless steel sieve. The juice may be sieved before the solids are removed or may be sieved after the solids have been removed.

The Genipa americana L used in step (i) of the method of the invention to extract the genipin or the Genipa americana L extract comprising genipin, may be green (i.e. unripe) and/or 10 cm or less long. For example, the Genipa americana L may be green and from about 5 cm to about 10 cm long and optionally, has a pH below 4.0.

By the term “unripe” we mean that the genipa used in step (i) is:
a) harvested at least one week before maturity, such as at least two weeks before maturity. For example, the genipa is harvested from about four weeks to about one week before maturity and/or
b) the firmness of the fruits is from MFP values >10 N to MFP values <2 N. MFP values >10 N are considered as unripe fruits, and fruits with MFP values <2 N are considered as ripe (MFP values measured by determining the maximum force of penetration (MFP) using a texturometer) and/or
c) the content of genipin in the endocarp is more than 3.4 mg/g and/or
d) has essentially no geniposidic acid in the endocarp.

In step (i) the genipin (such as an extract obtained or obtainable from Genipa americana L comprising genipin) is contacted with oxygen.

Contacting the genipin (such as an extract obtained or obtainable from Genipa americana L comprising genipin) with oxygen allows for oxidation to occur.

The genipin (such as an extract obtained or obtainable from Genipa americana L comprising genipin) may be contacted with oxygen using any suitable means and/or in any suitable container.

For example, the genipin (such as an extract obtained or obtainable from Genipa americana L comprising genipin) may simply be exposed to oxygen in the atmosphere to enable oxidation to occur, i.e., placed in a container which is open to the atmosphere.

By the term “exposed” we mean that at least 0.1%, or at least 1%, or at least 10% of the surface of the extract is in contact with oxygen in the atmosphere.

The genipin (such as an extract obtained or obtainable from Genipa americana L comprising genipin) may be exposed to oxygen in the atmosphere (i.e., allow oxidation) for at least 10 minutes, such as at least 20 minutes or at least 30 minutes. For example, the extract may be left open to the atmosphere from about 10 minutes to about 40 hours, such as from about 20 minutes to about 20 hours or from about 30 minutes to about 10 hours.

In certain embodiments oxygen or a mixture of gases with oxygen may be injected to the extract to accelerate the oxidation step.

During this time, the genipin (such as an extract obtained or obtainable from Genipa americana L comprising genipin, i.e. juice) may change colour from brown to blue.

Typically, the genipin (such as an extract obtained or obtainable from Genipa americana L comprising genipin) is exposed to oxygen in the atmosphere with no additional solvent being added. However, solvents that may be added include, but are not limited to water and/or ethanol.

The genipin (such as an extract obtained or obtainable from Genipa americana L comprising genipin) may be exposed to oxygen in the atmosphere at a temperature of from about 20° C. to about 70° C., such as from about 20° C. to about 50° C. or from about 20° C. to about 40° C. It has been found that above 70° C., the colour of the extract may change to black.

The pH of the extract may be from about 4.0 to about 7.0.

In step (ii) of the method of the invention, the genipin (such as an extract obtained or obtainable from Genipa americana L comprising genipin) of step (i) is combined with a composition comprising amino acids, polypeptides and/or proteins including but not limited to an extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides, and/or proteins.

In certain embodiments, the composition comprising amino acids, polypeptides and/or proteins has a concentration of amino acids, polypeptides and/or proteins of at least 5% w/w, such as at least 10%, such as at least 20%, such as at least 50%, such as at least 70% w/w.

Typically, the extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and/or proteins is a vegetal extract (i.e. an extract obtained from a vegetable source) that is high in protein. High in protein in the present invention is understood as having at least at least 20% w/w of proteins, such as at least 30% w/w of proteins.

In the method of the invention, it may be preferred that the CCP, such as an extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and/or proteins, is white or colourless.

For example, the CCP may be a white bean extract, a pea extract, oat extract, soy extract or a milk extract.

The extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides, and/or proteins may be obtained using any extraction solvent. For example, the solvent may be water (aqueous solvent), alcohol or a mixture of water and alcohol (hydro-alcoholic solvent).

For example, the extract obtained or obtainable from fruits, vegetables, grains, legumes, nuts, seeds, plant materials, animal materials including milk and eggs, microbial and algal materials, by-products from such sources and mixtures thereof may be an aqueous extract, an alcohol extract (which includes hydro-alcoholic extracts) or an organic extract.

The term “aqueous extract” as used herein, refers to the extract obtained or obtainable from fruits, vegetables, grains, legumes, nuts, seeds, plant materials, animal materials including milk and eggs, microbial and algal materials, by-products from such sources and mixtures thereof when the extraction from the plant has been performed using water as the only solvent.

The term “alcohol extract” as used herein, refers to the extract obtained or obtainable from fruits, vegetables, grains, legumes, nuts, seeds, plant materials, animal materials including milk and eggs, microbial and algal materials, by-products from such sources and mixtures thereof when the extraction from the plant has been performed using an alcohol as the solvent. The alcohol solvent may consist of only alcohol (e.g. 100% alcohol), for example 100% ethanol, or may be a mixture of an alcohol and water (i.e. a hydro-alcoholic solvent), for example, a mix of ethanol and water (hydro-ethanolic solvent), for example, from about 1% to about 99% alcohol (e.g. ethanol) in water, for example the ratio of water to alcohol is from 10/90% v/v to 90/10% v/v or 30/70% v/v to 70/30% v/v, such as 50/50% v/v or 70/30 v/v.

The term “organic extract” as used herein, refers to the extract obtained or obtainable from fruits, vegetables, grains, legumes, nuts, seeds, plant materials, animal materials including milk and eggs, microbial and algal materials, by-products from such sources and mixtures thereof when the extraction has been performed using an organic solvent that is not an alcohol. For example, the organic solvent may be selected from the group consisting of acetic acid, acetone, acetonitrile, benzene, 2-butanone, carbon tetrachloride, chlorobenzene, chloroform, cyclohexane, 1,2-dichloroethane, diethylene glycol, diethyl ether, diglyme (diethylene glycol, dimethyl ether), 1,2-dimethoxy-ethane (glyme, DME), dimethyl-formamide (DMF), dimethyl sulfoxide (DMSO), 1,4-dioxane, ethyl acetate, ethylene glycol, glycerin, heptane, hexamethylphosphoramide (HMPA), hexamethylphosphorous, triamide (HMPT), hexane, methyl t-butyl, ether (MTBE), methylene chloride, N-methyl-2-pyrrolidinone (NMP), nitromethane, pentane, petroleum ether (ligroine), pyridine, tetrahydrofuran (THF), toluene, triethyl amine, o-xylene, m-xylene, p-xylene and mixtures thereof.

In a preferred aspect, the extract obtained or obtainable from fruits, vegetables, grains, legumes, nuts, seeds, plant materials, animal materials including milk and eggs, microbial and algal materials, by-products from such sources and mixtures thereof is an aqueous extract.

As will be appreciated by the person skilled in the art, as used herein the term “obtainable from” means that the extract may be obtained from a plant/animal or may be isolated from the plant/animal, or may be obtained from an alternative source, for example by chemical synthesis or enzymatic production. Whereas the term “obtained” as used herein, means that the extract is directly derived from the plant/animal source.

The quantity of solvent used may be 4 to 10 times the quantity of fruits, vegetables, grains, legumes, nuts, seeds, plant materials, animal materials including milk and eggs, microbial and algal materials, by-products from such sources and mixtures thereof. For example, the ratio of solvent to fruits, vegetables, grains, legumes, nuts, seeds, plant materials, animal materials including milk and eggs, microbial and algal materials, by-products from such sources and mixtures thereof may be from about 4:1 to about 10:1.

The extract obtained or obtainable from fruits, vegetables, grains, legumes, nuts, seeds, plant materials, animal materials including milk and eggs, microbial and algal materials, by-products from such sources and mixtures thereof may typically be obtained by extraction at a temperature of 50° C. or less. For example, at a temperature of from about 10° C. to about 50° C., such as from about 20° C. to about 40° C.

Before extraction, the fruits, vegetables, grains, legumes, nuts, seeds, plant materials, animal materials including milk and eggs, microbial and algal materials, by-products from such sources and mixtures thereof may be dried and/or ground to generate particles. The particles may be from about 0.01 cm to about 1 cm, such as 0.1 cm to about 0.5 cm.

Any means known to the skilled person can be used to grind the fruits, vegetables, grains, legumes, nuts, seeds, plant materials, animal materials including milk and eggs, microbial and algal materials, by-products from such sources and mixtures thereof. For example, the fruits, vegetables, grains, legumes, nuts, seeds, plant materials, animal materials including milk and eggs, microbial and algal materials, by-products from such sources and mixtures thereof may be ground by using hammer or knives and passing through a sieve, such as a 19 mm sieve.

In step (ii) of the method of the invention, the product of step (i) and a composition comprising amino acids, polypeptides and/or proteins as defined above, may be combined in a weight ratio of from about 0.5:4 to about 0.5:40 genipin/protein, polypeptide and/or amino acids, such as from about 1:4 to about 1:30 genipin/protein, polypeptide and/or amino acids.

The inventors of the present invention have surprisingly and unexpectedly found that the ratio in which the product of step (i) and the composition comprising amino acids, polypeptides and/or proteins (or CCP) as defined above are combined has an effect on the colour provided by the resulting composition and/or colourant. Thus a stable blue and a stable black colour may be obtained using different ratios between the genipin and the proteins, polypeptides and/or amino acids.

For example, where the ratio of genipin in the product from step (i) to the protein, peptide and/or polypeptide of the CCP (such as the proteins from a protein rich extract obtained from white beans) is from about 0.5:10 to about 1:60, such as from about 0.5:10 to about 0.5:50, such as from about 0.5:10 to about 1:30, the resulting colour provided by the composition is blue, whereas where the ratio of genipin contained in the product from step (i) to the protein, peptide and/or polypeptide of the CCP (such as the proteins from a protein rich extract obtained from white beans) is from about 0.5:5 to about 1:10, such as from about 0.5:4 to about 0.5:8 the colour provided by the colourant/composition is black.

For the ratios described herein between the genipin and the protein, polypeptide and/or amino acid, the quantity of genipin is measured before the oxidation reaction of step (i).

In certain embodiments, the CCP is a white bean extract (for example a white bean extract comprising at least 20% w/w of proteins, polypeptides and/or amino acids), and the ratio of genipin in the product from step (i) to the white bean protein, peptide and/or polypeptide is of 0.6:13 or more, or of 0.6:15 or more (such as 0.6:20 or more or 0.6:30 or more), the resulting colour provided by the composition is blue, whereas where the ratio of genipin contained in the product from step (i) to the protein, peptide and/or polypeptide of the white beans extract is of 0.6:15 or less, or of 0.6:13 or less, such of 0.6:10 or less, or such as 0.6:6 or less, the colour provided by the colourant/composition is black.

In certain embodiments, the CCP is an oat extract (for example an oat extract comprising at least 2.4% w/w of proteins, polypeptides and/or amino acids), and the ratio of genipin in the product from step (i) to the oat protein, peptide and/or polypeptide is of 0.6:10 or more, or of 0.6:12 or more (such as 0.6:20 or more or 0.6:30 or more), the resulting colour provided by the composition is blue, whereas where the ratio of genipin contained in the product from step (i) to the protein, peptide and/or polypeptide of the oat extract is of 0.6:12 or less; or of 0.6:10 or less, such as 0.6:8 or less, or such as 0.6:6 or less, the colour provided by the colourant/composition is black.

In certain embodiments, the CCP is a pea extract (for example comprising at least 5% w/w proteins of proteins, polypeptides and/or amino acids), and the ratio of genipin contained in the product from step (i) to the pea protein, peptide and/or polypeptide is of 0.6:20 or more, or of 0.6:25 or more (such as 0.6:30 or more or 0.6:40 or more), the resulting colour provided by the composition is blue, whereas where the ratio of genipin contained in the product from step (i) to the protein, peptide and/or polypeptide of the pea extract is 0.6:25 or less; or 0.6:20 or less, such as 0.6:15 or less, or such as 0.6:10 or less, the colour provided by the colourant/composition is black.

In certain embodiments, the CCP is milk (for example comprising at least 3.4% w/w proteins of proteins, polypeptides and/or amino acids), and the ratio of genipin contained in the product from step (i) to the milk protein, peptide and/or polypeptide is of 0.6:15 or more, or of 0.6:17 or more (such as 0.6:20 or more or, 0.6:30 or more), the resulting colour provided by the composition is blue, whereas where the ratio of genipin contained in the product from step (i) to the protein, peptide and/or polypeptide of the milk is 0.6:17 or less; or 0.6:15 or less, such as 0.6:10 or less, or such as 0.6:8 or less, the colour provided by the colourant/composition is black.

The product of step (i) and the CCP, such as an extract obtained or obtainable from fruits, vegetables, grains, legumes, nuts, seeds, plant materials, animal materials including milk and eggs, microbial and algal materials, by-products from such sources and mixtures thereof, may be combined for a time period of from about 1 hour to about 10 hours, such as from about 3 hours to about 5 hours.

The product of step (i) and the CCP, such as an extract obtained or obtainable from fruits, vegetables, grains, legumes, nuts, seeds, plant materials, animal materials including milk and eggs, microbial and algal materials, by-products from such sources and mixtures thereof may be combined in any suitable container, with or without suitable stirring/agitation. For example, the combining in step (ii) may be done with continuous stirring and/or agitation.

The product of step (i) and the CCP, such as an extract obtained or obtainable from fruits, vegetables, grains, legumes, nuts, seeds, plant materials, animal materials including milk and eggs, microbial and algal materials, by-products from such sources and mixtures thereof may be combined at temperature of from about 20° C. to about 70° C., such as from about 30° C. to about 60° C. or from about 35° C. to about 55° C.

The pH of the combination in step (ii) about may be from about 4 to about 6, such as from about 5 to about 6 or from about 5.3 to about 5.6.

Optionally, in step (iii) of the method of the invention, the product obtained in step (ii) may be filtered to remove any solid material (bagasse). For example, the product of step (ii) may be filtered through a filter press or hydraulic press with a sieve, such as a 200 mesh polyester mesh.

The present inventors have also surprisingly and unexpectedly found that, combining the product of step (i) with an alkali earth metal hydroxide, such as calcium hydroxide, provides a colourant/composition that provides a stable brown colour.

Thus, the present invention also provides a method for providing a brown colourant/composition comprising:
(i) Contacting genipin (such as an extract obtained or obtainable from Genipa americana L comprising genipin) with oxygen;
(ii) Combining the product from step (i) with an alkali earth metal hydroxide, such as calcium hydroxide; and optionally
(iii) filtering the product obtained in step (ii) to remove insoluble solids.

For each part of genipin (such as the genipin comprised in an extract obtained or obtainable from Genipa americana L, such as a genipap juice), 0.002 to 0.01 parts of alkali earth metal hydroxide, such as calcium hydroxide are used.

In the composition of the final brown colour, the proportion of the alkali earth metal hydroxide, such as calcium hydroxide is from 1.0 to 2.0%.

In each of the methods described above, the filtered mixture (filtrate) obtained after step (iii) may be optionally concentrated. The filtrate obtained may be concentrated using any suitable means, such as a Bernauer evaporator or a Falling film evaporator or reverse osmosis.

Therefore, the present invention also provides a method of providing a composition and/or colourant (in particular, a composition that provides a blue or black colour or a blue or black colourant), wherein the method comprises:
(i) Contacting genipin (such as an extract obtained or obtainable from Genipa americana L comprising genipin) with oxygen;
(ii) Combining the product from step (i) and a CCP, such as an extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and/or proteins; optionally
(iii) Filtering the mixture obtained in step (ii) to remove solids; and optionally
(iv) Concentrating the product obtained in step (ii) or (iii).

Or, a method for providing a brown colourant/composition comprising:
(i) Contacting genipin (such as an extract obtained or obtainable from Genipa americana L comprising genipin) with oxygen;
(i) Combining the product from step (i) with an alkali earth metal hydroxide, such as calcium hydroxide; optionally
(iii) Filtering the mixture obtained in step (ii) to remove solids; and optionally
(iv) Concentrating the product obtained in step (ii) or (iii).

The filtrate may be concentrated at a temperature of from about 30° C. to about 90° C., such as from about 50 to about 80° C.

Depending on the form of the composition and/or colourant provided by the method of the invention, the concentrated filtrate obtained in step (iii) or (iv) may be combined with excipients.

Alternatively, the concentrated filtrate may be the composition and/or the colourant.

For example, where the composition and/or colourant is in the form of a solid, such as a powder, in the method of the invention, the concentrated filtrate obtained in step (iii) or (iv) may be mixed with a solid excipient, such as maltodextrin (corn or cassava), arabic gum, dextrin, inulin, starch, rice flour, cashew gum, cassava starch, or mixtures thereof. The mixture may be homogenized to ensure complete dissolution of the excipient.

Where the concentrated filtrate is mixed with a solid excipient, the concentrated filtrate may be mixed with the solid excipient at a temperature of from about 50 to about 90° C., such as from about 60 to about 80° C.

Where the filtrate is mixed with solid excipients, the composition and/or colourant may comprise from about 30 to about 90%, such as from about 40 to about 80% by weight extracts, from about 10 to about 70%, such as from about 20 to about 60% weight excipient.

After being mixed with solid excipients, the solid mixture may then be sterilized and/or dried.

Where the solid mixture is sterilized, the mixture is heated at a temperature of from about 50 to about 150° C., such as from about 60 to about 125° C.

Where the solid mixture is dried, the mixture is dried using spray drying, vacuum drying, a fluidized bed, a lyophilizer or any other techniques known in the art that provide a similar effect. Typically, the mixture is dried at a temperature of from about 100 to about 250° C., such as from about 130 to 200° C.

After the solid mixture has been sterilized and/or dried, the resulting solid may be used as a colourant/colourant composition.

After drying, the product may comprise from about 1 to about 10% water, such as from about 2 to about 6% water by weight of the composition and/or colourant.

Where the composition and/or colourant is in the form of a liquid, in the method of the invention, the concentrated filtrate may be mixed with a liquid excipient, such as glycerin, propylene, glucose syrup, polysorbate or mixtures thereof. The mixture may be homogenized to ensure complete dissolution of the excipient.

Where the filtrate is combined with liquid excipients, the concentrated filtrate may be mixed with the liquid excipient at a temperature of from about 50 to about 90° C., such as from about 60 to about 80° C.

In the method of the invention, where the filtrate is combined with liquid excipients, the composition and/or colourant may comprise from about 30 to about 90%, such as from about 40 to about 80% by weight extracts, from about 10 to about 70%, such as from about 20 to about 60% weight excipient.

The liquid mixture may then be sterilized. Where the liquid mixture is sterilized, the mixture is heated at a temperature of from about 50 to about 150° C., such as from about 60 to about 125° C. After sterilization the liquid may be used as a colourant/composition.

The method of the invention as described above provides a stable blue colourant/composition, in particular a blue colourant/composition that is stable to heat.

The method of the invention as described above also provides a stable black colourant/composition, in particular a black colourant/composition that is stable to heat.

The method of the invention as defined above also provides a stable brown colourant/composition, in particular, a brown colourant/composition that is stable to heat.

The present invention also provides a blue, black or brown colourant/composition, in particular a stable blue, black or brown colourant/composition obtained by the methods defined herein.

### Compositions and Colourants

As explained above, the present inventors have surprisingly found that a composition comprising genipin (such as an extract obtained or obtainable from Genipa americana L) and a composition comprising amino acids, polypeptides and/or proteins (or CCP) including but not limited to an extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and proteins, provides a stable colourant, for example, a stable blue or black colourant. In particular, a blue or black colourant that is stable to heat.

Therefore, the present invention provides a composition comprising genipin (such as an extract obtained or obtainable from Genipa americana L) and a composition comprising amino acids, polypeptides and/or proteins including but not limited to an extract obtained or obtainable from fruits, vegetables, grains, legumes, nuts, seeds, plant materials, animal materials including milk and eggs, microbial and algal materials, by-products from such sources and mixtures thereof, wherein the composition provides a stable blue or black colourant.

In particular, a composition comprising an extract obtained or obtainable from Genipa americana L and an extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and proteins, wherein the composition provides a stable blue or black colourant. For example, a blue or black colourant that is stable to heat.

Such compositions are hereinafter referred to as the “blue composition of the invention” or the “black composition of the invention”

The present invention also provides a natural colourant comprising an extract obtained or obtainable from genipa and a composition comprising amino acids, polypeptides and/or proteins such as an extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and proteins, wherein the natural colourant provides a stable blue or black colourant. For example, a blue or black colourant that is stable to heat.

Such colourants are hereinafter referred to as the “blue colourant of the invention” or the “black colourant of the invention.”

In the composition and/or colourant of the invention, the extract obtained or obtainable from genipa is as defined in the method of the invention above, and the extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and proteins is also as defined in the methods of the invention above.

The composition and/or colourants of the invention may be in the form of a liquid or a solid.

Where the composition and/or colourant provides a blue colour, i.e. a blue colourant, the composition and/or colourant may comprise genipin (such as an extract obtained or obtainable from Genipa americana L) and an CPP such as an extract obtained or obtainable from white beans, pea, oat and/or milk.

Therefore, the present invention provides a composition that provides a blue colour or a blue colourant, wherein the composition and/or colourant may comprise an extract obtained or obtainable from genipa and an extract obtained or obtainable from white beans, pea, oat and/or milk.

Where the composition provides a blue colour or is a blue colourant, the blue composition and/or blue colourant may be obtained using a method as defined above, wherein the ratio of product from step (i) to the CCP, such as an extract as defined before is from about 0.5:10 to about 1:60, such as from about 0.5:10 to about 0.5:50, such as from about 0.5:10 to about 1:30. Where the composition that provides a blue colour or a blue colourant is obtained by a method as defined above, the CCP may be an extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and proteins, in particular, the extract may be obtained or obtainable from white beans, pea, oat and/or milk.

Where the composition provides a black colour or is a black colourant, the black composition and/or black colourant may be obtained using a method as defined above, wherein the ratio of product from step (i) to the CCP, such as an extract as defined before is from about 0.5:5 to about 1:10, such as from about 0.5:4 to about 0.5:8. Where the composition that provides a black colour or a black colourant is obtained by a method as defined above, the CCP may be an extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and proteins, in particular, the extract may be obtained or obtainable from white beans, pea, oat and/or milk.

Therefore, the present invention provides a composition that provides a black colour or a black colourant, wherein the composition and/or colourant may comprise an extract obtained or obtainable from genipap and an extract obtained or obtainable from fruits, vegetables, grains, legumes, nuts, seeds, plant materials, animal materials including milk and eggs, microbial and algal materials, by-products from such sources and mixtures thereof, which contain amino acids, polypeptides and proteins. In a preferred embodiment, the sources are beans, pea, oat, soy, milk and mixture thereof.

As explained above, the present inventors have also found that combining an extract obtained or obtainable from genipap and an alkali earth metal hydroxide, such as calcium hydroxide, a composition that provides a brown colour or a brown colourant is provided (or brown colorant of the invention).

Therefore, the present invention provides a composition that provides a brown colour or a brown colourant, wherein the brown composition and/or brown colourant may comprise genipin (such as an extract obtained or obtainable from genipap) and an alkali earth metal hydroxide, such as calcium hydroxide.

As already mentioned before in the methods of the invention, the genipin is oxidized previously to be combined with the alkali earth metal hydroxide, such as calcium hydroxide.

Where the composition provides a brown colour or is a brown colourant, the composition and/or colourant may be obtained using a method as defined above, wherein for one part product from step (i) (such as a unripe genipap juice), 0.001 to 1.01 parts of alkali earth metal hydroxide, such as calcium hydroxide is used.

The present method of production of a stable brown colour obtained or obtainable from genipap does not need of further stabilization with proteins or amino acids.

Thus, in certain embodiments, the method of production of a stable brown colorant does not needs the addition of a protein or amino acid source to provide a stable brown colorant.

In certain embodiments, the stable brown colorant of the invention do not comprises an endogenous natural source of proteins or amino acids.

In certain embodiments, the final product (stable brown colorant) comprises an alkali earth metal hydroxide, such as calcium hydroxide at a concentration from about 1.0 to 2.0% w/w of the brown colorant composition.

Genipin and the extract obtained or obtainable from genipap that comprises genipin are as defined above for the methods of the invention.

An advantageous aspect of the present invention is that the composition and/or colourant may be prepared using a very simple process with minimal steps. As will be appreciated by the person skilled in the art, simplification and/or reduction of process steps is advantageous as it reduces process time, costs and/or the need to specific equipment to perform the process.

Unless otherwise stated herein, the weight percentages listed are based on the total weight of (dry) composition obtained.

For the avoidance of doubt, preferences, options, particular features and the like indicated for a given aspect, feature or parameter of the invention should, unless the context indicates otherwise, be regarded as having been disclosed in combination with any and all other preferences, options particular features and the like as indicated for the same or other aspects, features and parameters of the invention.

The term “about” as used herein, e.g. when referring to a measurable value (such as an amount or weight of a particular component in the reaction mixture), refers to variations of +20%, +10%, +5%, +1%, +0.5%, or, particularly, +0.1% relative to the specified amount. For example, a variation of +0.5% with regards to the percentage of a component in the composition of the invention, means a variation of 0.5% relative to the percentage given, i.e. +0.5% of 10% would mean a variation from 9.5% to 10.5%.

For the avoidance of doubt, in this specification when we use the term “comprising” or “comprises” we mean that the extract or composition being described must contain the listed ingredient(s) but may optionally contain additional ingredients. When we use the term “consisting essentially of” or “consists essentially of” we mean that the extract or composition being described must contain the listed ingredient(s) and may also contain small (for example up to 5% by weight, or up to 1% or 0.1% by weight) of other ingredients provided that any additional ingredients do not affect the essential properties of the extract or composition. When we use the term “consisting of” or “consists of” we mean that the extract or composition being described must contain the listed ingredient(s) only.

It is also intended that the terms “comprise” or “comprises” or “comprising” may be replaced with “consist” or “consisting” or “consisting essentially of” throughout the application as required.

Food acceptable ingredients include those known in the art (including those also referred to herein as pharmaceutically acceptable excipients) and that can be natural or non-natural, i.e. their structure may occur in nature or not. In certain instances, they can originate from natural compounds and be later modified (e.g. maltodextrin).

As noted above, the present invention provides a stable colourant/composition, such as a stable blue, brown or black colourant/composition.

Thus, the present invention also provides a composition and/or colourant as defined above for use as a blue, brown or black colourant, i.e. a stable blue, brown or black colourant.

The present invention also provides the use of a composition or colourant as defined above, to provide a blue, brown or black colourant, i.e. a stable blue, brown or black colourant.

By the term “stable”, we mean that the colour provided by the composition and/or colourant remains the same colour hue, L*a*b, for at least 1 day, such as at least 1 week or at least 1 month.

In certain embodiments, the colour (blue, black or brown) provided by said compositions is stable to heat, acidic/basic conditions and/or light exposure.

In the present invention, the term “stable to acidic conditions” means that the colour hue of the blue, brown or black colours of the present invention is not changed or has a change of less than 30%, such as less than 20%, such as less than 10%, or such as less than 5% of colour hue change in respect to the colour hue measured before the acidic treatment. “Acidic treatment or condition” means in the present application a pH of less than 5, such as less than pH 4.5, such as less than pH 4, such as less than pH 3, or such as less than pH 2.

In the present invention, the term “stable to basic conditions” means that the colour hue of the blue, brown or black colours of the present invention is not changed or has a change of less than 30%, such as less than 20%, such as less than 10%, or such as less than 5% of colour hue change in respect to the colour hue measured before the acidic treatment. “basic treatment or condition” means in the present application a pH of more than 7, such as more than pH 8, such as more than pH 9.

The colour (blue, black or brown) provided by the composition and/or colourant may stable to heat, i.e. is heat stable.

By the term “heat stable”, we mean that the colour provided by the composition and/or colourant remains the same colour hue, L*a*b, for at least 1 day, such as at least 1 week or at least 1 month either after being subjected to heat during processing, such as being heated in a cooked food product or being stored at elevated temperatures. In the present invention, the term “stable to heat” means that the colour hue provided by the at least one phycobilins (such as phycocyanin) is not changed or has a change of less than 30% in respect to the colour hue measured before the heat treatment, such as less than 20%, such as less than 10%, or such as less than 5% of colour hue change.

For example, the colour may remain the same colour hue, L*a*b, for at least 1 day, such as at least 1 week or at least 1 month after being heated during the formation of a food product at a temperature of from about 50 to about 250° C., such as from about 100 to about 200° C.

Thus, in certain embodiments, the colour provided by said compositions or colorants of the invention is stable after a thermal treatment of more than 40° C., such as a treatment of more than 50° C., 60° C., 70° C., 80° C., or such as more than 90° C. for a period at least 30 seconds, at least 1 minute, at least 5 minutes, at least 10 minutes, at least 30 minutes, at least 1 hours, at least 2 hours, or such as at least 5 hours.

In certain embodiments, the colour provided by said compositions or colorants of the invention is stable to light exposure.

In certain embodiments, the colour (blue, black or brown) provided by the composition or colorant of the invention is stable to light exposure.

In the present invention, the term “stable to light” or “stable to light exposure” means that the colour hue provided by the blue, black or brown colorant is not changed or has a change of less than 30% in respect to the colour hue measured at dark conditions.

Thus, in certain embodiments of the composition or complex of the invention, the blue, black or brown colour is stable after a light exposure of more than 5 minutes, such as more than 30 minutes, such as more than 1 hour, such as more than 2 hours, 3, hours, 4 hours, 5 hours, 10 hours, 12 hours, 24 hours, 2 days, 10 days, such as more than 30 days, or such as more than 60 days.

The present invention also relates to the method as defined before for stabilising the blue or black colour of genipap extract.

Thus, the present invention also provides the use of a composition (blue, black and/or brown composition) and/or colourant (blue, black or brown colourant) as defined above to colour a consumable, a cosmetic, a pharmaceutical or nutraceutical preparation.

### Uses

The present invention is also related to a consumable, a cosmetic or a pharmaceutical or nutraceutical preparation comprising a composition and/or colourant as defined above.

In one aspect, use of a composition and/or colourant as defined above in colouring a food product is provided.

The composition and/or colourant as defined above can be added to a food product in an amount effective to increase, enhance and/or modify the colour characteristics of a food product or portion thereof.

Food encompasses the following general food categories, as defined by the Food and Drug Administration (FDA): baked goods and baking mixes, including all ready-to-eat and ready-to-bake products, flours, and mixes requiring preparation before serving; beverages, alcoholic, including malt beverages, wines, distilled liquors, and cocktail mix; beverages and beverage bases, non-alcoholic, including only special or spiced teas, soft drinks, coffee substitutes, and fruit and vegetable flavored gelatin drinks; breakfast cereals, including ready-to-eat and instant and regular hot cereals; cheeses, including curd and whey cheeses, cream, natural, grating, processed, spread, dip, and miscellaneous cheeses; chewing gum, including all forms; coffee and tea, including regular, decaffeinated, and instant types; condiments and relishes, including plain seasoning sauces and spreads, olives, pickles, and relishes, but not spices or herbs; confections and frostings, including candy and flavored frosting, marshmallows, baking chocolate, and brown, lump, rock, maple, powdered, and raw sugars; dairy product analogs, including nondairy milk, frozen or liquid creamers, coffee whiteners, toppings, and other nondairy products; egg products, including liquid, frozen, or dried eggs, and egg dishes made therefrom, i.e., egg roll, egg foo young, egg salad, and frozen multicourse egg meals, but not fresh eggs; fats and oils, including margarine, dressings for salads, butter, salad oils, shortenings and cooking oils; fish products, including all prepared main dishes, salads, appetizers, frozen multicourse meals, and spreads containing fish, shellfish, and other aquatic animals, but not fresh fish; fresh eggs, including cooked eggs and egg dishes made only from fresh shell eggs; fresh fish, including only fresh and frozen fish, shellfish, and other aquatic animals; fresh fruits and fruit juices, including only raw fruits, citrus, melons, and berries, and home-prepared “ades” and punches made therefrom; fresh meats, including only fresh or home-frozen beef or veal, pork, lamb or mutton and home-prepared fresh meat-containing dishes, salads, appetizers, or sandwich spreads made therefrom; fresh poultry, including only fresh or home-frozen poultry and game birds and home-prepared fresh poultry-containing dishes, salads, appetizers, or sandwich spreads made therefrom; fresh vegetables, tomatoes, and potatoes, including only fresh and home-prepared vegetables; frozen dairy desserts and mixes, including ice cream, ice milks, sherbets, and other frozen dairy desserts and specialties; fruit and water ices, including all frozen fruit and water ices; gelatins, puddings, and fillings, including flavored gelatin desserts, puddings, custards, parfaits, pie fillings, and gelatin base salads; grain products and pastas, including macaroni and noodle products, rice dishes, and frozen multicourse meals, without meat or vegetables; gravies and sauces, including all meat sauces and gravies, and tomato, milk, buttery, and specialty sauces; hard candy and cough drops, including all hard type candies; herbs, seeds, spices, seasonings, blends, extracts, and flavorings, including all natural and artificial spices, blends, and flavors; jams and jellies, home-prepared, including only home-prepared jams, jellies, fruit butters, preserves, and sweet spreads; jams and jellies, commercial, including only commercially processed jams, jellies, fruit butters, preserves, and sweet spreads; meat products, including all meats and meat containing dishes, salads, appetizers, frozen multicourse meat meals, and sandwich ingredients prepared by commercial processing or using commercially processed meats with home preparation; milk, whole and skim, including only whole, lowfat, and skim fluid milks; milk products, including flavored milks and milk drinks, dry milks, toppings, snack dips, spreads, weight control milk beverages, and other milk origin products; nuts and nut products, including whole or shelled tree nuts, peanuts, coconut, and nut and peanut spreads; plant protein products, including the National Academy of Sciences/National Research Council “reconstituted vegetable protein” category, and meat, poultry, and fish substitutes, analogs, and extender products made from plant proteins; poultry products, including all poultry and poultry-containing dishes, salads, appetizers, frozen multicourse poultry meals, and sandwich ingredients prepared by commercial processing or using commercially processed poultry with home preparation; processed fruits and fruit juices, including all commercially processed fruits, citrus, berries, and mixtures; salads, juices and juice punches, concentrates, dilution, “ades”, and drink substitutes made therefrom; processed vegetables and vegetable juices, including all commercially processed vegetables, vegetable dishes, frozen multicourse vegetable meals, and vegetable juices and blends; snack foods, including chips, pretzels, and other novelty snacks; soft candy, including candy bars, chocolates, fudge, mints, and other chewy or nougat candies; soups, home-prepared, including meat, fish, poultry, vegetable, and combination home-prepared soups; soups and soup mixes, including commercially prepared meat, fish, poultry, vegetable, and combination soups and soup mixes; sugar, white, granulated, including only white granulated sugar; sugar substitutes, including granulated, liquid, and tablet sugar substitutes; and sweet sauces, toppings, and syrups, including chocolate, berry, fruit, corn syrup, and maple sweet sauces and toppings.

For example, the composition and/or colourant as defined above can be used to impart a blue colour similar to the colour obtained when using Brilliant Blue FCF (artificial color) to food products, such as dairy, confectionery, beverages, sauces/gravies, etc.

A dairy product may refer to yogurt, custard, milk smoothie, milk shake, dairy ice cream.

A confectionery product may refer to a sweet or candy food product, such as chewing gums or hard and soft confectionery products. Non-limiting examples of confectionery products include cakes, cookies, pies, chocolates, chewing gums, gelatins, ice creams, puddings, jams, jellies, gummies, hard boiled candies, chewy candies, cereal and other breakfast foods, canned fruits and fruit sauces.

A beverage product may refer to beverages, beverage mixes and concentrates, including but not limited to alcoholic and non-alcoholic ready to drink and dry powdered beverages. Non-limiting examples of beverages can include carbonated and non-carbonated beverages, e.g., sodas, fruit or vegetable juices.

A sauce product may refer to a sweet or savoury semi-solid composition used to add flavour, moisture and/or visual appeal to a dish. Non-limiting examples of sauces include gravy and barbecue sauce.

A meat analogue product may refer to foods made from vegetarian ingredients, which approximate certain aesthetic qualities (such as texture, flavour, appearance) or chemical characteristics of specific types of meat. Non-limiting examples of meat analogues include vegetable (veggie) burgers.

In yet another aspect, it is provided a food product comprising a food colouring composition as described hereinbefore. For example, the food product may be as described above.

In certain embodiments, the food product may be bakery products (i.e. cakes, breads, croissants, pies, cheese breads, sweet breads, cookies, biscuits, cereal bars, breakfast cereals, pop corn), dairy products (i.e. ice cream, milk, yogurt, cheeses and other possible dairy products), drink products (i.e. fruit juices, water, alcoholic drinks, such as whiskey, gin, vodka, beer), confectionary products (i.e. chocolates, sweets, gums, confectionery, and other confectionery products) and general food products (i.e. pasta, pastries, lasagna and other possible pasta).

Thus, the present invention also provides blue, brown or black foodstuffs, such as bakery products (i.e. cakes, breads, croissants, pies, cheese breads, sweet breads, cookies, biscuits, cereal bars, breakfast cereals, pop corn), dairy products (i.e. ice cream, milk, yogurt, cheeses and other possible dairy products), drink products (i.e. fruit juices, water, alcoholic drinks, such as whiskey, gin, vodka, beer), confectionary products (i.e. chocolates, sweets, gums, confectionery, and other confectionery products) and general food products (i.e. pasta, pastries, lasagna and other possible pasta)) comprising a composition and/or colourant as defined above.

One of ordinary skill in this art will recognize that the optimal amount of food colouring composition present in a given food product is determined by factors such as overall desired colour, solubility, regulatory approval, etc. One of ordinary skill in this art can readily determine the optimal amount of colour for a given product based on those factors.

In one aspect, use of composition and/or colourant as defined above in colouring a pharmaceutical, cosmeceutical, nutraceutical or cosmetic product is provided. The composition and/or colourant as defined above can be added to the product (pharmaceutical, nutraceutical or cosmetic product) in an amount effective to increase, enhance and/or modify the colour characteristics of the product or a portion thereof.

In another aspect, the invention relates to a pharmaceutical composition, cosmeceutical, a nutraceutical or to a cosmetic composition comprising composition and/or colourant as defined above and, optionally a vehicle adequate for the formulation of said composition and/or colourant as defined above into said pharmaceutical composition, cosmeceutical or cosmetic composition.

The stabilized composition and/or colourant as defined above would provide a desired colour (such as a stable blue colour, brown or black colour) to said pharmaceutical, cosmeceutical, cosmetic or nutraceutical formulation.

“Pharmaceutical composition”, as used herein, relates to compositions and molecular entities that are physiologically tolerable. Preferably, the term “pharmaceutically acceptable” means it is approved by a regulatory agency of a state or federal government or is included in the U.S. Pharmacopoeia or other generally recognized pharmacopoeia for use in animals, and more particularly in humans.

“Cosmetic composition”, as used herein refers to a composition suitable for use in personal hygiene of human beings or animals, or in order to enhance the natural beauty or change the body appearance without affecting the structure or functions of the human or animal body, comprising one or more products providing such effects. If desired, the cosmetic composition provided by the invention can contain, in addition to the composition of the invention, one or more cosmetics or cosmetic products, i.e., substances or mixtures intended to be placed in contact with the external parts of the human or animal body (e.g., epidermis, hair system, nails, lips, etc.) or with the teeth and the buccal mucosa, for the exclusive or main purpose of cleaning them, perfuming them, changing their appearance, protecting them, keeping them in good condition or correcting body odors. Illustrative examples of cosmetically acceptable vehicles include the products contained in the INCI (International Nomenclature of Cosmetic Ingredients) list. The composition and/or colourant as defined above may be added to a wide variety of products for cosmetic application, including makeup, creams for cleansing, protecting, treating, or caring for the skin, in particular, the face, hands, and feet (e.g., day and night creams, makeup removal creams, foundation creams and sunscreens), liquid foundations, makeup removal lotions, protective or skin-care body lotions, sunscreen lotions, skin care lotions, gels, or foams, such as cleansing, sunscreen, and artificial tanning lotions, bath preparations, deodorant compositions, after-shave gels or lotions, depilatory creams, and compositions used for insect stings and against pain. The composition of the invention may take any of a wide variety of forms, and include, for example dressings, lotions, solutions, sprays, creams, gels, ointments, or the like.

As used herein, the term “cosmeceutical product” refers to a product suitable for use in the body or animal body comprising one or more cosmeceutical products (functional cosmetics, dermaceuticals or active cosmetics), i.e., topical hybrid products with cosmetic-pharmaceutical characteristics containing active ingredients having effect on user's skin, hair and/or nails, at higher and more effective concentrations, therefore they are located in an intermediate level between cosmetic and drug. Illustrative examples of cosmeceutical products include essential oils, ceramides, enzymes, minerals, peptides, vitamins, etc.

As used herein, the term “nutraceutical product” refers to a product suitable for use in human beings or animals, comprising one or more natural products with therapeutic action which provide a health benefit or have been associated with disease prevention or reduction, and it includes dietary supplements presented in a non-food matrix (e.g., capsules, powder, etc.) of a concentrated natural bioactive product usually present (or not) in the foods and which, when taken in a dose higher than that existing in those foods, exerts a favorable effect on health which is greater than effect which the normal food may have. Therefore, the term “nutraceutical product” includes isolated or purified food products as well as additives or food supplements which are generally presented in dosage forms normally used orally, for example, capsules, tablets, sachets, drinkable phials, etc.; such products provide a physiological benefit or protection against diseases, generally against chronic diseases. If desired, the nutraceutical product provided by the invention can contain, in addition to the composition of the invention, one or more nutraceuticals (products or substances associated with disease prevention or reduction), for example, flavonoids, omega-3 fatty acids, etc., and/or one or more prebiotics (non-digestible food ingredients which stimulate probiotic activity and/or growth), for example, oligofructose, pectin, inulin, galacto-oligosaccharides, lactulose, human milk oligosaccharides, dietary fiber, etc.

In certain embodiments, the product (consumable or food product, pharmaceutical, cosmeceutical, nutraceutical or cosmetic product) has a pH of less than 6, such as less than 5 pH, such as less than 4, such as less than 3, or such as less than 2.

In certain embodiments, the product (consumable or food product, pharmaceutical, cosmeceutical, nutraceutical or cosmetic product) has been treated with a thermal treatment.

The consumable (food product), cosmetic, pharmaceutical or nutraceutical preparation may comprise the composition (blue, black and/or brown composition) and/or colourant (blue, black or brown colourant) as defined above in a concentration of from about 0.1% to about 10%, such as from 0.5% to 1%.

As explained previously, the use of a composition and/or colourant that comprises both an extract obtained or obtainable from genipa and an extract obtained or obtainable from white beans, soy, pea, chickpea, lentil or mixtures thereof provides a colour that is surprisingly and unexpectedly stable, i.e. heat stable compared to a blue colour provided by an extract from genipa alone.

Thus, the present invention provides the use of an extract obtained or obtainable from white beans, soy, pea, chickpea, lentil or mixtures thereof (as previously defined) in stabilising the blue colour of genipa extract (as previously defined).

As used herein, the term “stabilising” is as defined for stable above.

The present invention, therefore, provides a method of producing a stable blue colourant, wherein the method comprises combining an extract obtained or obtainable from genipa (as previously defined) and an extract obtained or obtainable from white beans, soy, pea, chickpea, lentil or mixtures thereof (as previously defined).

The stable blue colourant may be heat stable. The stable blue colorant may be stable to neutral and low pH (such as a pH below 4, or below 3 or below 2). The stable blue colorant may be stable to high pH (such as more than 7, such as more than 8, such as more than 10).

### BRIEF DESCRIPTION OF FIGURES

FIG. 1 shows the stability of the blue colourant (A), black colourant (B) and brown colourant (C) at different pH.

FIG. 2 shows bread cooked without colorant (1), blue colorant (2 and 3), brown colorant (4) and black colorant (5).

FIG. 3 shows cakes prepared with blue colorant and black colorant.

FIG. 4 shows a cake prepared using the brown colorant.

FIG. 5 shows brown colorant light stability vs brown colorants known in the art.

FIG. 6 shows plant base hamburgers coloured with the brown colorant and black colorant of the invention and with other brown colorants known in the art.

### EXAMPLES

The present invention will be further described by reference to the following, non-limiting examples.

### Example 1—Preparation of a Genipap Extract

Whole, unripe (green) Genipap fruits are pressed to obtain the juice. The juice is sieved through a 30-60 mesh stainless steel sieve and the solids (bagasse) are discarded.

### Fruit Ripeness Stage:

For the ripeness stage determination, two batches containing 25 fruits each were selected, including both the unripe genipap, with 150 g and 20 cm of diameter, and the ripe fruits, with 230 g and 25 cm of diameter, respectively. The fruits ripeness stage was determined based on the instrumental colour parameters, firmness, physicochemical characteristics and sugar profile.

### Firmness Analysis

The firmness was measured by determining the maximum force of penetration (MFP) by using a texturometer (Stable MicroSystems, model TA-XT Plus, Surrey, UK) equipped with a P/4N needle probe. The MFP was determined on two opposite faces in the equatorial region of each fruit and the results were expressed in newtons (N). Fruits with MFP values >10 N were considered unripe, and fruits with MFP values <2 N were considered ripe, as described previously by Bentes, A. S. et al. (Bioactive compounds of genipap, orange and mango: composition and development of extraction methods, Campinas, Brazil, 2014).

The Genipap extract (0.67% w/w of genipin) is placed in an open container to allow oxidation of the juice. The juice is left in the open container for from about 20 minutes to about 40 hours or until the juice changes colour from brown to dark blue, at a temperature of from about 20° C. to about 50° C.

### Example 2—Preparation of an Extract Rich in Proteins from White Bean Extract

White beans are ground using hammer and knives and then passed from a 19 mm sieve to generate a particle of 0.1 cm to 0.5 cm.

The ground white beans are then added to water (approximately 4 to 10 times the amount of the white beans) and extracted with water for from about 10 minutes to about 50 minutes at a temperature of from about 20° C. to about 50° C.

The final % of proteins in the white protein extract is of 22% w/w proteins.

### Example 3—Preparation of a Crude Blue Colourant

To get the blue colour, the Genipin contained in the oxidized juice obtained from the green fruit of Genipap (example 1) is mixed to react with the proteins, polypeptides and/or amino acids contained in white bean extract, soy extract (water extraction 5-10:1), oat extract (water extraction 5-10:1), pea extract (water extraction 5-10:1), and milk.

1 part of oxidized genipap juice (0.67% w/w genipin) was mixed with 0.7 parts white beans extract of example 2 (22% w/w proteins) from about 2 hours to about 5 hours at a temperature of from about 35° C. to about 55° C. using constant agitation.

The ratio of genipin/white beans proteins is of 0.67:15.

When the amount of white bean proteins is decreased from this, the resulting colour is black.

Other natural extracts containing proteins may be also sued to obtain a stable blue. For example an oat extract containing 2.4% w/w proteins, a pea extract containing 5% w/w of proteins or a soy extract containing 38% w/w proteins were mixed with the oxidiced genipap juice and a stable blue color was obtained:

1 part of oxidized genipap juice (0.67% w/w genipin) was mixed with 5 parts oat extract (2.4% w/w proteins) from about 2 hours to about 5 hours at a temperature of from about 35° C. to about 55° C. using constant agitation.

The ratio of genipin/oat beans proteins is of 0.67:12.

When the amount of oat proteins is decreased from this, the resulting colour is black.

1 part of oxidized genipap juice (0.67% w/w genipin) was mixed with 5 parts pea extract (5% w/w proteins) from about 2 hours to about 5 hours at a temperature of from about 35° C. to about 55° C. using constant agitation.

The ratio of genipin/oat beans proteins is of 0.67:25.

When the amount of pea proteins is decreased from this, the resulting colour is black. 1 part of oxidized genipap juice (0.67% w/w genipin) was mixed with 0.3 parts of soy extract (38% w/w proteins) from about 2 hours to about 5 hours at a temperature of from about 35° C. to about 55° C. using constant agitation. The ratio of genipin/soy proteins is of 0.67:11.4.

When the amount of soy proteins is decreased from this, the resulting colour is black.

Animal proteins were also tested such as milk containing 3.4% w/w proteins:

1 part of oxidized genipap juice (0.67% w/w genipin) was mixed with 5 parts of milk (3.4% w/w proteins) from about 2 hours to about 5 hours at a temperature of from about 35° C. to about 55° C. using constant agitation.

The ratio of genipin/milk proteins is of 0.67:17.

When the amount of milk proteins is decreased from this, the resulting colour is black.

Further examples mixing one part of genipap juice (0.67% w/w genipin) with higher amounts of the above mentioned proteins extracts or milk were done and a stable blue colour was obtained. Thus, using higher ratios of protein vs genipin the colour obtained is still a stable blue.

The above mentioned mixtures where then filtered through a filter press or in a hydraulic press with a 200 mesh polyester mesh to remove solids and concentrated in a Bernauer evaporator, or Falling film evaporator or reverse osmosis at from about 50° C. to about 80° C. to provide a crude blue colourant.

### Example 4—Preparation of a Dry Final Blue Colourant

The products of Example 3 were heated at a temperature of from about 60° C. to about 80° C. and mixed with a solid excipient or blend of excipients (Maltodextrin (corn or cassava), or arabic gum, or dextrin, or inulin, or starch, or rice flour, or cashew gum, or cassava starch, or mixture thereof), in the proportion of min. 1 to max. 2 kg of concentrate to min. 0.2 kg to max 0.6 kg of excipient.

The mixtures were then homogenized until complete dissolution of excipients.

Finally, the mixtures were sterilized at a temperature of from about 60° C. to about 125° C., and then dried using spray drying, or a vacuum dryer, or a fluidized bed, or a lyophilizer.

### Example 5—Preparation of a Liquid Final Blue Colourant

The products of Example 3 were heated at a temperature of from about 60° C. to about 80° C. and mixed with a liquid excipient or blend of excipients (Glycerine, or propylene, or glucose syrup, or polysorbate or mixture thereof), in the proportion of min. 1 to max. 2 kg of concentrate to min. 0.2 kg to max 0.6 kg of excipient.

Finally, the mixtures were sterilized at a temperature of from about 60° C. to about 125° C.

### Example 6. Black Colour (Reaction of Genipin with Proteins)

A stable black colour can be also obtained from the unripe genipap juice extract of example 1 (oxidized juice). For this, the Genipin containing juice obtained from the green fruit of Genipap of example 1 is mixed to react with different protein containing natural extracts or with milk.

1 part of genipap juice from example 1 (0.67% w/w genipin) was mixed with 0.3 parts of white beans extract (22% w/w proteins) from about 2 hours to about 5 hours at a temperature of from about 35° C. to about 55° C. using constant agitation. The ratio of genipin/white bean proteins is of 0.67:6.6. The color obtained was a stable black.

1 part of genipap juice from example 1 (0.67% w/w genipin) was mixed with 2 parts of oat extract (2.4% w/w proteins) from about 2 hours to about 5 hours at a temperature of from about 35° C. to about 55° C. using constant agitation. The ratio of genipin/white bean proteins is of 0.67:4.8. The colour obtained was a stable black.

1 part of genipap juice from example 1 (0.67% w/w genipin) was mixed with 2 parts of pea extract (5% w/w proteins) from about 2 hours to about 5 hours at a temperature of from about 35° C. to about 55° C. using constant agitation. The ratio of genipin/white pea proteins is of 0.67:10. The colour obtained was a stable black.

1 part of genipa juice from example 1 (0.67% w/w genipin) was mixed with 2 parts of soy extract (38% w/w proteins) from about 2 hours to about 5 hours at a temperature of from about 35° C. to about 55° C. using constant agitation. The ratio of genipin/white bean proteins is of 0.67:19. The colour obtained was a stable black.

1 part of genipap juice from example 1 (0.67% w/w genipin) was mixed with 2 parts of milk (3.4% w/w proteins) from about 2 hours to about 5 hours at a temperature of from about 35° C. to about 55° C. using constant agitation. The ratio of genipin/milk proteins is of 0.67:6.8. The colour obtained was a stable black.

The mixtures obtained were dried similarly to what was described previously in example 4 or the mixture may be concentrated similarly to what was described previously in example 5. The result is a stable black colorant.

### Example 7. Brown Color

To get the brown colour, the Genipin containing juice obtained from the green fruit of Genipap of example 1 (oxidised) is mixed to react with calcium hydroxide according to the proportion in the table below:

TABLE 1

| Amount of Genipin in the Genipap juice | Amount of |
|---|---|
| (0.67% w/w genipin) | calcium hydroxide |
| 0.5 to 1.0 g | 0.002 to 0.005 g |

Then the mix was filtered. The filtrate was dried similarly to what was described previously in example 4 or the filtrate may be concentrated similarly to what was described previously in example 5. The result is stable brown colorants with a Lab as shown in Table 2.

TABLE 2
Lab and dE measurements of two batches
of brown colorants A and B

| Sample | L | A | b | dE00 |
|---|---|---|---|---|
| Powder A brown | 54.01 | 4.54 | 12.04 | |
| Powder B brown | 51.82 | 2.99 | 9.27 | 3.2 |
| 0.2% powder A in Water | 79.07 | 3.37 | 29.57 | |
| 0.23% powder B in water | 78.46 | 1.87 | 27.22 | 1.62 |
| Liquid (with glycose syrum) | 78.5 | 1.95 | 28.19 | 1.35 |
| 0.34% in water | | | | |
| Liquid (without glucose syrup) | 77.42 | 2.16 | 30.72 | 1.64 |
| 0.34% in water | | | | |

### Example 8—Bakery, Yogurt and Beverage Applications

8.1. PH stability

1% of blue colour obtained in example 4 was diluted in water.

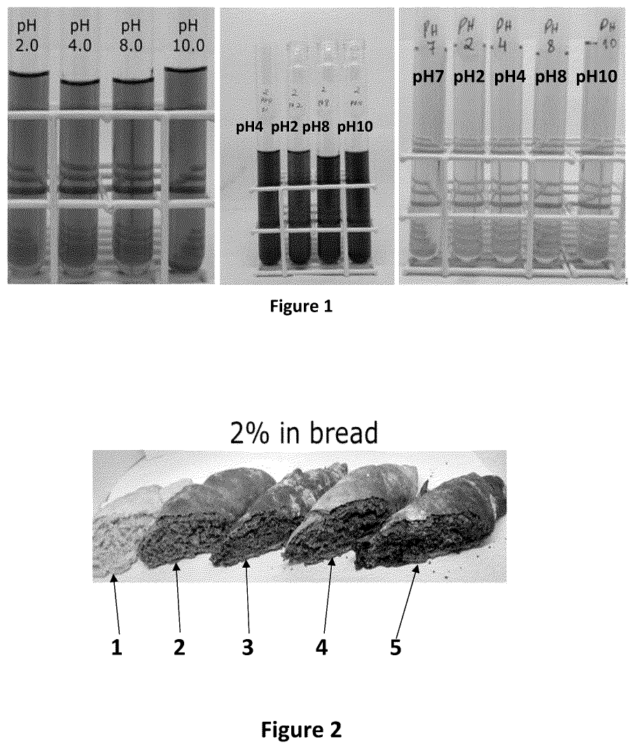

The colour is stable in pH 2, 4, 8 and 10 (see FIG. 1A).

1% of black colour of example 6 was diluted in water. The colour is stable at pH2, 4, 8 and 10.

0.1% of brown colour of example 7 was diluted in water. The colour is stable at pH7, 2, 4, 8 and 10.

The Lab of a dilution of 0.2% of blue colourant diluted in water at pH3 was measured. The colour was stable over time as it can be seen in tables 3 and 4.

TABLE 3
Lab measurements. Time 0, 15 minutes,
30 minutes and 60 minutes.

| | | Blue colour |
|---|---|---|
| Time | | 0.2% pH 3 |
| 0 | L | 84.87 |
| | a | −6.72 |
| | b | −10.87 |
| 15 | L | 82.96 |
| | a | −5.76 |
| | b | −11.50 |
| 30 | L | 82.40 |
| | a | −5.20 |
| | b | −11.34 |
| 60 | L | 81.84 |
| | a | −4.49 |
| | b | −10.78 |

TABLE 4
dE00 reading against time 0

| | Blue colour from Genipap |
|---|---|
| Time | 0.2%, pH 3 |
| 15 | 1.7 |
| 30 | 2.33 |
| 60 | 3.07 |

### 8.2 Heat Stability of the Blue Colorant

0.2% of the blue colorant was dissolved in water at pH neutral or in water at pH3 and the resulting solutions were heated up to 80° C. in water bath.

Lab was measured at time 0, 15, 30 and 60 minutes. Blue color was stable over time (Table 5)

TABLE 5
Lab measurements.

| 0.2% DI Water | | | | | 0.2% pH 3 | | | | |
|---|---|---|---|---|---|---|---|---|---|
| | L | a | b | dE00 | | L | a | b | dE00 |
| 0 | 83.34 | −7.59 | −12.61 | | 0 | 84.87 | −6.72 | −10.87 | |
| 15 | 80.71 | −6.39 | −13.85 | 2.34 | 15 | 82.96 | −5.76 | −11.50 | 1.70 |
| 30 | 80.10 | −6.06 | −13.73 | 2.82 | 30 | 82.40 | −5.20 | −11.34 | 2.33 |
| 60 | 79.45 | −5.40 | −12.69 | 3.43 | 60 | 81.84 | −4.49 | −10.78 | 3.07 |

Time 0, 15 minutes, 30 minutes and 60 minutes.

### 8.3. Colorant in Hydroalcoholic Application.

1% of blue color obtained in example 4 or 5 was diluted in cachaça (Sugarcane liquor).

Color stability: 3 months

### 8.4. Colorant in Bakery Products.

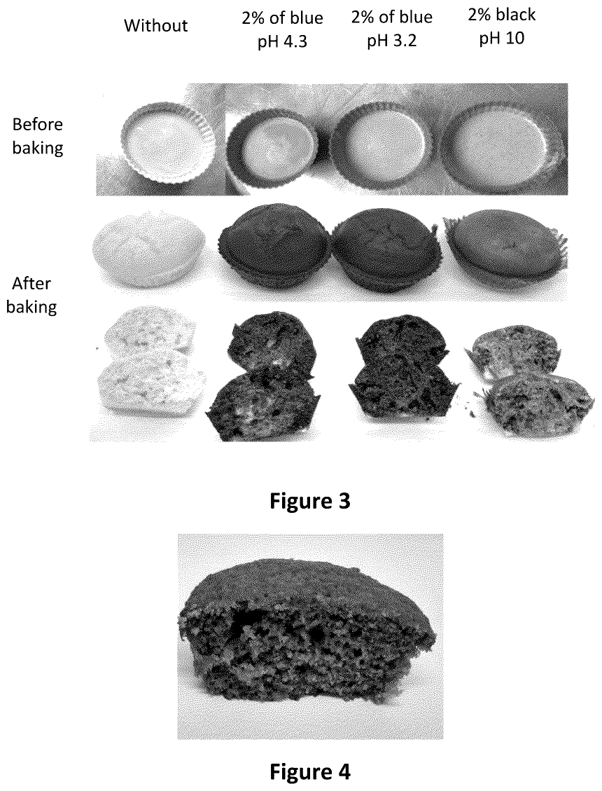

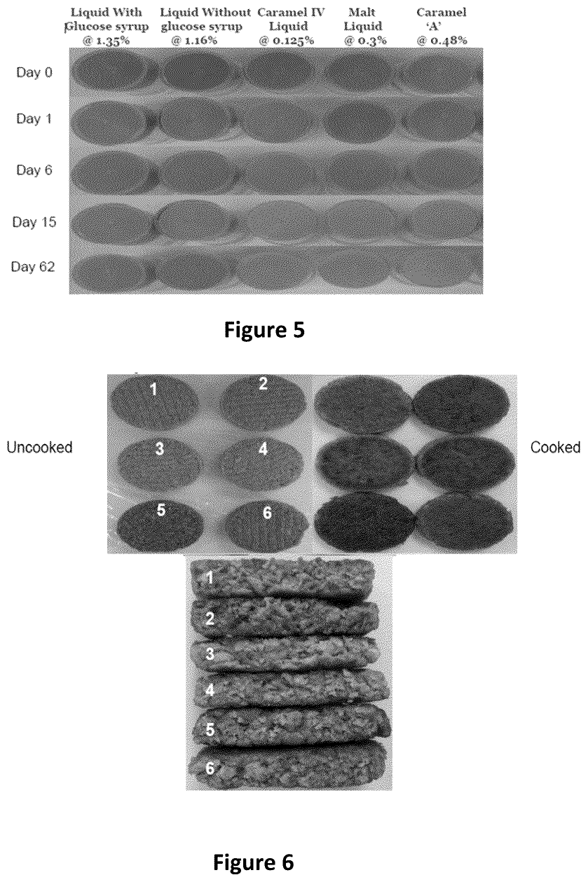

Cake: Base cake recipe (for 15 cupcakes with 10 g each after baking): 3 eggs, 150 ml of water, 60 grams of sugar, 100 grams of flour, 5 g of oil, 1 g of yeast, 2 g of dry or concentrated blue, black or brown extract (FIGS. 3 and 4) Blue, brown and black colours were stable after cooking.

### 8.5. Pop Corns.

After the process of popping the corn forming the popcorn, 100 g of ready-made popcorn are mixed (manually in a plastic bag) 1 g of dry blue colorant.

Color stability: 2 months

### 8.6. Colorant in Dairy Products: Ice Cream, Milk, Yogurt, Cheeses and Other Possible Dairy Products.

In the commercial yogurt base (purchased in the market), 5 and 10% of dry or concentrated blue, brown or black colorant was added, mixed by hand.

Color stability: 2 months

### 8.7. Colorant in Pasta.

In the water used to cook the pasta, 1% dry blue colorant is added, kept at a boil at 100° C., after cooking for 8 to 10 minutes the pasta changed color to blue (purple blue).

Color stability: 1 month (products without preservatives)

### 8.8. Colorant in Bread.

Base bread recipe (for 20 breads with 50 g each after baking): 1 egg, 250 ml of water, 10 grams of sugar, 1 kg of flour, 15 g of oil, 50 g of yeast, 10 g of dry or concentrated blue, brown or black colorant. Blue, brown and black colours were stable after cooking (FIG. 2)

### Example 9

Application in creams and body lotion.

1% of the blue colorant, brown colorant or black colorants were added to a liquid soap base.

1% of the blue colorant, brown colorant or black colorants were added to a body lotion base.

Blue, black and brown colors were stable after 6 months.

### Example 10. Brown Color Light Stability

For light stability, sugar paste bought from local supermarket was used. Samples were dosed as indicated in Table 6. Samples were placed in small pots and then read on the spectrocolorimeter (Konica Minolta CM-3600A). Samples were then read at regular intervals. Samples were compared against day 0 and the dE00 value was obtained (table 6). The greater the dE00, the greater the deviation in shade. As it can be observed from table 6, the guanipapo extracts are much more stable to light if compare with other standard brown colors present on the market (caramel iv, Caramel A and Malt)

TABLE 6

| | dE00 | | | | |
|---|---|---|---|---|---|
| | | Guanipapo | | | |
| | Guanipapo | Liquid | | | |
| | brown | without | | | |
| | Liquid with | glucose | | | |
| | glucose | syrup | Caramel IV | | Caramel |
| | syrup | @ | Liquid @ | Malt liquid | ‘A’ |
| Day | @ 1.35% | 1.35% | 0.125% | @ 0.3% | @ 0.48% |
| 1 | 0.82 | 0.92 | 1.19 | 0.60 | 1.82 |
| 6 | 2.31 | 2.44 | 3.48 | 3.54 | 4.55 |
| 15 | 3.58 | 3.70 | 4.67 | 5.51 | 6.47 |
| 62 | 3.50 | 3.27 | 3.61 | 3.86 | 7.43 |

### Example 11, Brown Colour and Black Colour in Meat Analogue

Soy plant based hamburgers were coloured using the brown and black colours of the invention and compared with malt and caramelised pear. FIG. 6 shows the plant based hamburgers before and after cooking (from outside and from the inside).
1—Brown genipapo color powder 1%
2—Black genipapo color powder 1%
3—Brown genipapo color liquid (glucose syrup) 1%
4—Brown genipapo color liquid (without glycose syrup) 1%
5—Malt liquid 1%
6—Caramelised Pear 1%

1. A method for providing a colourant/composition comprising:
(i) contacting genipin with oxygen to form a product;
(iia) combining the product from step (i) with an alkali earth metal hydroxide, and optionally (iii); or
(iib) combining the product from step (i) with a composition comprising amino acids, polypeptides, and/or proteins; and optionally (iii)
(iii) filtering the product obtained in step (iia) or (iib) to remove insoluble solids.

2. The method for providing a composition/colourant of claim 1, wherein the method comprises:
wherein the product from step (i) and the protein, peptide and/or polypeptide of the composition comprising amino acids, polypeptides, and/or proteins from step (iib) is combined in a ratio of from about 0.5:5 to about 1:10, and the colour provided by the composition and/or colourant is black.

3. The method of claim 2, wherein the composition and/or colourant is a stable black colourant is heat stable, stable to acidic and/or basic conditions, and/or stable to light exposure

4. The method according to claim 3, wherein the product of step (i) and the composition comprising amino acids, polypeptides, and/or proteins are combined for a time period of from about 1 hour to about 10 hours.

5. The method according to claim 4, wherein the product of step (i) composition comprising amino acids, polypeptides, and/or proteins may be is combined at temperature of from about 20° C. to about 70° C.

6. The method according to claim 2, wherein the composition comprising amino acids, polypeptides, and/or proteins is an extract obtained from fruits, vegetables, grains, legumes, nuts, seeds, plant materials, animal materials, microbial and algal materials, by-products from such sources and mixtures thereof, obtained using water, alcohol or a mixture of water and alcohol.

7. The method according to claim 2, wherein the genipin is contacted with oxygen at a temperature of from about 20° C. to about 70° C.

8. The method according to claim 2, wherein the genipin is an extract obtained from Genipa americana L.

9. The method according to claim 8, wherein the extract obtained from Genipa americana L is from unripe Genipa americana L.

10. The method according to claim 8, wherein the Genipa americana L extract comprises at least 0.5% w/w genipin.

11. The method according to claim 2, wherein (iv) the filtered mixture obtained after step (iii) is concentrated.

12. The method according to claim 11, wherein the filtrate obtained in step (iii) or the concentrated filtrate (iv) is mixed with a solid excipient.

13. The method according to claim 11, wherein the filtrate obtained in step (iii) or the concentrated filtrate (iv) is mixed with a liquid excipient.

14. A black colourant/composition obtained by the method of claim 2.

15. A brown colourant/composition obtained by the method of claim 1.

16. A black colourant comprising genipin and a composition comprising amino acids, polypeptides, and/or proteins combined in a ratio of from about 0.5:5 to about 1:10.

17. A colourant according to claim 16, wherein the composition comprising amino acids, polypeptides, and/or proteins is obtained from white beans, peas, soy, oat and/or milk.

18. A brown colourant, comprising genipin and an alkali earth metal hydroxide.

19. The colorant according to claim 16, wherein the genipin is oxidised.

20. The colorant according to claim 19, wherein the genipin is an extract obtained from Genipa americana L, optionally from unripe Genipa americana L.

21. (canceled)

22. The colorant according to claim 20, wherein the black colourant is heat stable, stable to acidic and/or basic pHs, and/or stable to light exposure.

23. The use according to claim 21 or 22, the colour A method of colouring consumable or food products, pharmaceutical, cosmeceutical, nutraceutical, or cosmetic products with the black colourant of claim 20.

24. A consumable or food product, pharmaceutical, cosmeceutical, nutraceutical, or cosmetic product comprising the black colourant or composition according to claim 14.

25. The colorant according to claim 18, wherein the genipin is oxidised.

26. The colorant according to claim 25, wherein the genipin is an extract obtained from Genipa americana L, optionally from unripe Genipa americana L.

27. The colorant according to claim 26, wherein the brown colourant is heat stable, stable to acidic and/or basic pHs, and/or stable to light exposure.

28. A method of colouring consumable or food products, pharmaceutical, cosmeceutical, nutraceutical, or cosmetic products with the brown colourant of claim 27.

29. A consumable or food product, pharmaceutical, cosmeceutical, nutraceutical, or cosmetic product comprising the brown colourant or composition according to claim 15.
